(19) United States
(12) Patent Application Publication
(10) Pub. No.: US 2007/0049794 A1
(43) Pub. Date: Mar. 1, 2007

(54) Visualization stylet for medical device applications having self-contained power source

(75) Inventors: Raymond Glassenberg, Wilmette, IL (US); Zebadiah Kimmel, Brookline, MA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: EZC Medical LLC

(21) Appl. No.: 11/217,743

(22) Filed: Sep. 1, 2005

(51) Int. Cl. A61B 1/04; A61B 1/06

(52) U.S. Cl. 600/109; 600/179; 600/130; 600/118

(57) ABSTRACT

A visualization stylet suitable is provided for use with medical devices to illuminate and visualize the interior anatomy of a body cavity or organ, wherein the stylet includes a miniature camera, light source and on-board power source.

### FIELD OF THE INVENTION

The present invention relates to medical devices and methods used to illuminate and visualize the interior anatomy of a body cavity or organ using a miniature camera having an on-board power source. The present invention has particular applicability to visualization of the interior of the oral cavity during endotracheal intubation.

### BACKGROUND OF THE INVENTION

In the course of providing medical care, particularly in an emergent situation or during anesthesia, it is frequently necessary to insert a tube into a patient's trachea to allow anesthesia and/or for the mechanical ventilation of the lungs of the patient. This procedure is called endotracheal intubation. It is important that the endotracheal tube be placed into the patient's trachea, rather than into the patient's esophagus (or anywhere else), otherwise air will not be delivered to the lungs. For this reason, it is important to be able to visualize the patient's glottis during endotracheal intubation. Improper endotracheal intubation is a significant cause of morbidity and mortality during anesthesia.

Typically, a device called a laryngoscope is used to facilitate endotracheal intubation. This device consists of a handle and a blade. There is the straight blade (“Miller blade”), and the slightly curved blade (“Macintosh blade”). The epiglottis normally overlies the glottic opening into the larynx to prevent the passage of food into the trachea during eating; therefore, in endotracheal intubation, it is necessary to displace the epiglottis from the glottic opening to permit the endotracheal tube to be inserted into the trachea. The blade is inserted into the patient's mouth and is used to lift the patient's tongue and epiglottis out of the way so that the patient's glottis (the entrance to the trachea) may be visualized, and the endotracheal tube may be inserted successfully into the trachea.

In some patients, such as obese patients or patients with atypical anatomy, the laryngoscope alone is unable to provide a clear view of the patient's glottis. So-called “blind intubation” may be attempted in such patients, but the failure rate of blind intubation is high. Blind intubation frequently leads to trauma and bleeding of the mucosa of the larynx and successful intubation often may require several attempts, slowing critical care and jeopardizing the patient's health. In such patients, therefore, it is desirable to place a visualization and/or an illumination device into the patient's pharynx to provide for a view of the glottis and allow proper insertion of the endotracheal tube.

In some cases, oral intubation is not desirable or practicable and a nasal intubation must be used. Three main techniques are used for nasal intubation. One method is to use an oral laryngoscope to observe and monitor the placement of the nasal endotracheal tube. A second method is to use blind intubation, manipulating the tube and/or the patient's head and neck. A third method employs flexible fiber-optic bronchoscope to both guide and visually confirm the proper placement of the endotracheal tube. Regardless of whether oral or nasal intubation is performed, the glottis must eventually be negotiated, and so illumination and visualization are highly desirable either way.

Several visualization and/or illumination devices have been produced and are in commercial use. For example, Aaron Medical Industries produces a lighted intubation guide that is essentially a thin stylet (wand) having a bright light at the tip. This stylet is placed within the endotracheal tube. As the endotracheal tube with its contained stylet is guided (blindly) through the pharynx, the operator of the tube may judge the approximate location of the tip of the endotracheal tube by observing the location of light transmitted through the patient's neck. This device provides no direct visualization of the glottis, and supplies only a small improvement over blind intubation.

Volpi Corporation manufactures a stylet device that provides direct visualization of the glottis. The device is placed within the endotracheal tube and uses fiber-optic bundles to transmit visual information from the tip of the tube back to the operator of the device. This device does not have its own light source, but requires a separate light source. Another endotracheal intubation device and methods of using it is described in a paper by Hikaru Kohase “Endotracheal Intubation Device with a Charge Couple Device Camera” (Anesth. Analg. 2003: 96: 432-434). The device comprises a wand with a Charge Couple Device (CCD) camera mounted at the distal end, and includes a side tube through which a tube introducer is inserted. The introducer is positioned into the trachea through the vocal cords, and the wand is then withdrawn, leaving the introducer in place. Vitaid Airway Management Corporation sells the GlideScope™ device, which embeds a video camera and a Light Emitting Diode (LED) light source within a laryngoscopic blade. This device does not fit inside an endotracheal tube.

Olympus and Pentax corporations both produce flexible fiber-optic bronchoscopes that include both image-transmitting and light-transmitting fiber-optic bundles, as well as a fully articulated and guidable tip. These devices, while originally designed for bronchoscopy (the visualization of the lung bronchi) may also be used for difficult intubations as follows. First, the distal portion of the fiber-optic bronchoscope is inserted within an endotracheal tube. Then, by looking through an eyepiece on the bronchoscope while manipulating the endotracheal tube, the operator of the device is able to directly visualize the placement of the tube within the trachea. When the endotracheal tube is successfully placed, the fiber-optic device is withdrawn from the tube. Used in this way, the fiber-optic bronchoscope is referred to as a fiber-optic laryngoscope (not to be confused with the blade-like regular laryngoscope described earlier). Use of such fiber-optic devices provides a considerable improvement over blind intubation, but these devices are very complex and expensive, and require extensive training for effective use.

The patent literature includes a number of devices for insertion into the oral cavity that provide illumination and visualization. One of the earliest examples is Smit U.S. Pat. No. 1,246,339, which discloses a tongue depressor having an internal electric light source and a glass light-conducting element that allows light to be conducted from the bulb to the tip of the instrument to aid in visualization of the oral cavity.

U.S. Pat. No. 6,655,377 to Pacey describes an endotracheal intubation instrument having a camera and a light positioned near the tip of the instrument. The camera and light may be powered by a battery internal to the handle of the device. The camera is optionally a CCD or CMOS (Complementary Metal Oxide Semiconductor) camera and the light source is optionally an LED. Suction is provided near the tip of the device to cool the light source and to remove moisture that would otherwise cloud the camera lens. The visualization elements are not designed to fit within an endotracheal tube, but are mounted outside and adjacent to a tube.

U.S. Pat. No. 6,652,453 to Smith describes a self-contained, light-weight laryngoscope that includes a digital camera and “light emitters” both positioned close to the distal end of the scope, powered by an internal battery. The device includes a clamp at the end that grasps the endotracheal tube to be guided into place. As above, this device is not designed to fit within an endotracheal tube.

U.S. Pat. No. 6,322,498 to Gravenstein describes a tracheal imaging scope with a CCD camera and an LED light positioned at the proximal end of the instrument (near the operator) and uses fiber-optics to transmit light and images between the distal end of the instrument and the camera/light. Simple electrical and/or optical “quick-connectors” are used to link the components and the camera and light(s) are powered by an external power source. The device may include a lumen for ventilation, irrigation or suction, but is not designed to fit within an endotracheal tube.

U.S. Pat. No. 5,842,973 to Bullard describes a self-contained nasal-endotracheal intubation device with an “optical channel” connected to a camera and a “light channel” connected to an internal light source. Power is supplied by an internal battery. This device may be placed within an endotracheal tube and used to guide it into place.

U.S. Pat. No. 3,677,262 to Zukowski describes an illuminated endotracheal tube inserter with a light source and fiber-optic viewing bundle. This inserter device is designed to fit within an endotracheal tube.

U.S. Pat. No. 5,329,940 to Adair describes a hand-held endotracheal tube insertion device that includes fiber-optic cables for transmitting light and images. The device includes a malleable “insertion section” and in use, a standard endotracheal tube is fitted over the insertion section and removably attached to the handle of the device to allow visualization and insertion of the endotracheal tube into the trachea. An inflatable cuff, of a type that is standard on most endotracheal tubes, is provided near the distal end of the device which, when in use, is inflated to seal the endotracheal tube in the trachea and properly position the tip of the tube above and between the two bronchi.

U.S. Pat. No. 4,337,761 to Upsher describes a laryngoscope with a curved blade that removably grasps an endotracheal tube. The blade additionally possesses a light source and a fiber-optic viewing member to permit visualization of the epiglottis and larynx. Power is supplied by a battery in the handle. The blade can be flexible so that it may be bent into various curvatures suitable to the anatomy or a particular patient.

U.S. Pat. No. 5,676,598 to Rudischhauser describes a laryngoscope with a curved spatula blade where the blade includes a waveguide for transmitting light and a separate image waveguide for transmitting images.

U.S. Pat. No. 6,629,924 to Adydelotte describes an “enhanced endotracheal tube” with a fiber-optic light bundle and a reflectively coated bore used to transmit images to the user. Additionally, an air passage is provided for inflating an inflatable cuff for positioning the device.

U.S. Pat. No. 6,146,402 to Munoz describes an endotracheal tube guide introducer that can be used to introduce a flexible guide tube into the trachea. Once in place, the guide tube is used to guide an endotracheal tube to its target. The device includes a fiber-optic visualization path as well as a light path for illuminating and viewing the epiglottis and larynx during use.

U.S. Pat. No. 5,665,052 to Bullard is another patent that describes an endotracheal tube guide. The guide is positioned in the trachea and an endotracheal tube is advanced along the guide to the desired location. Fiber-optic cables provide transmission of light and images.

U.S. Pat. No. 4,086,919 to Bullard discloses a laryngoscope for endotracheal intubation having a housing containing a working channel for containing forceps and channels containing fiber optics for lighting and viewing the internal areas of the body, and a laryngoscope blade for manipulating the epiglottis of a patient to enable viewing of a target area.

U.S. Pat. No. 3,766,909 to Ozbey describes a laryngoscope with a disposable blade and light guide. The light guide is incorporated into the blade and transmits light from a bulb in the handle. The bulb is powered by a battery, also located in the handle. The blade is designed to be cheap to manufacture and to be optionally disposable.

Visualization stylets, endotracheal guides and fiber-optic laryngoscopes and bronchoscopes were originally designed for bronchoscopy (visualization of the bronchi of the lungs), not for endotracheal intubation, and they generally suffer from a number of disadvantages. They often are complex and expensive to manufacture, requiring specialized parts fabrication and assembly. Due to their cost, they are generally non-disposable, which means that they have to be sterilized and carefully maintained after each use. This adds to the cost of maintaining such a device. They are generally difficult to sterilize due to the number and complexity of their sub-components and may require special procedures for cleaning and sterilization. They frequently are fragile, and fiber-optic light bundles are especially susceptible to damage. Repair is costly and takes the instrument out of use.

One of the main problems in the use of the fiber-optic laryngoscope/bronchoscope is a reflection of one of its benefits, that is, its flexibility. Because of its flexibility and complicated controlling system, it is often difficult to control the bronchoscope as it is advanced through the patient airways to the vocal cords. The proper use of such fiber-optic devices requires significant training and it is estimated that 25 to 50 practice intubations on a mannequin followed by 50 to 100 intubations on normal patients is required before a physician should attempts what is termed “difficult airway management.”

In view of the foregoing disadvantages, the financial cost of endotracheal intubation in patients who cannot be intubated solely through use of a regular laryngoscope (e.g., obese patients) is very high. In addition, significant delays in treatment may be caused by the need to locate and mobilize appropriate fiber-optic equipment. A need exists for a small, hand-held endotracheal visualization stylet that provides high quality optics and that is both easy to use and inexpensive to manufacture.

### SUMMARY OF THE INVENTION

In view of the foregoing, it is an object of the present invention to provide apparatus for visualizing a bodily cavity comprising a visualization stylet including a self-contained power source. The visualization stylet of the invention may be used for various medical procedures including endotracheal intubation or to visualize the internal features of any anatomical structure such as the colon, vagina, uterus, esophagus, nasal passages, ear passages, joints, or abdominal cavity.

In a preferred embodiment, the visualization stylet of the invention is used to facilitate endotracheal intubation. The visualization stylet is shaped and sized so that it may fit inside an endotracheal tube designed for endotracheal intubation of a human or animal subject. The stylet is elongated and preferably curved, and comprises a number of elements including a thin, flexible tube-shaped body defining a lumen therethrough, having a proximal end (near the operator) and a distal end (further away from the operator). The stylet also includes an image-gathering device, such as a charged couple device (CCD) or a complementary metal oxide semiconductor (CMOS) or a very large scale integrated (VLSI) chip camera, at or near the distal tip of the body, and a light-emitting device such as an LED or plurality of LEDs, also at or near the distal tip of the body. Electronic connectors transfer power and/or data to and from the image-gathering and light-emitting devices.

In use, the visualization stylet is placed within an endotracheal tube, such that the tip of the stylet is at the distal tip of the endotracheal tube, and the electronic connectors of the stylet are accessible from the proximal end of the endotracheal tube. The visualization stylet may optionally be reversibly attached in place relative to the endotracheal tube by using a standard luer-lock feature. The electronic connectors are attached to an internal or external power supply, as well as a visualization device such as a cathode ray tube (CRT) or equivalent device (e.g. an Liquid Crystal Display (LCD) monitor), thus providing a view of the patient's pharynx, glottis, and other anatomical structures during intubation. Once intubation is accomplished, the visualization stylet is withdrawn from the endotracheal tube and either sterilized for re-use, or preferably discarded.

The light source may be of any acceptable type; for example, it may be an incandescent electric light or preferably a light emitting diode (LED). The light source is generally mounted at the distal end of the stylet and is preferably positioned and shielded in such a way that the illumination from the light source does not interfere with the image received by the camera. In one embodiment, the light source is positioned in front of the camera and is shielded from the camera (for example, by the rim of a collimator) so that the light projects forward from the device and not backward towards the camera. Light may optionally be supplied by a light source separate from the device, wherein the light is transmitted to the distal tip of the stylet by means of fiber-optic cables. The stylet may employ a single light source such as an LED or a plurality of LEDs. Such LEDs may optionally be arranged in a generally circular pattern about the distal tip of the stylet.

The camera may be any suitable image collecting device known in the art, for example a charged couple device (CCD), complementary metal oxide semiconductor (CMOS), or other electronic camera may be used. The image received by the camera may be transmitted directly from the illuminated object or may be transmitted and focused from the illuminated object to the camera via a lens (or plurality of lenses).

Additionally, an optional collimator may be positioned in front of the lens. One or more LEDs may be mounted peripherally to the collimator, so that the collimator shields the camera from the light emitted by the LEDs. The collimator both improves the optics of the system by filtering non-parallel incoming light, and shields the camera from direct illumination by the light source(s).

In an alternative embodiment the stylet is provided with more than one camera. In particular, the provision of two adjacent cameras enables stereoscopic imaging. In a stereoscopic embodiment where lenses are used, the device may include a lens for each camera. Each camera may have one or more associated lenses. Each camera may optionally have its own lens(es) and its own collimator.

In this disclosure the term “lens” includes any transparent cover, whether or not it can serve to focus light, and specifically includes transparent covers whose sole purpose is to protect the image-gathering device (e.g., a camera).

In order to keep the front lens of the camera free of condensation, fluids, mucus or other debris, the device may also include one or more of the following. It may include a moisture-removing element such as a heating element in thermal communication with the lens to keep the lens free of moisture or a vacuum or suction device. It may include a debris-removing element to remove solid or liquid debris, such as a vacuum or suction device, or a lens-washing element or an air-jet or water-jet device, or a mechanical wiper device. These components may be activated at the will of the operator to maintain a clear view. Such devices are well-known and may be adapted for use with the invention.

Additionally, the lens may be pre-treated with a hydrophilic or hydrophobic substance in order to help manage water, blood, or other substances that may be encountered during intubation. Likewise, the lens may be constructed of a hydrophilic or hydrophobic material.

In certain embodiments one or more working channels may also be included in the stylet. Such a working channel can receive a flexible guide member, which in use may be passed through the working channel and guided through the vocal chords into the trachea prior to introduction of an intubation apparatus into the subject. Alternatively, the working channel may be used to receive a catheter or may be used for suction, delivery of oxygen or other gases, or delivery of local and/or general anesthetics to the subject.

In another embodiment, the distal tip of the stylet may be controllable by the operator and may be pivoted in two or three dimensions to allow additional visualization of internal structures. Methods of achieving such manipulation are known and described for instance in U.S. Pat. Nos. 5,318,008 and 5,842,973.

The stylet tube of the invention may be made from any suitable material that is malleable such that it may be bent into a shape suitable for introduction into the anatomy of a particular space such as the oral cavity and larynx. Suitable materials for making the stylet tube are well known in the catheter art and include metals such as aluminum, plastics and polymers such as polyvinylchloride, polypropylene, polyethylene, polyester, polyamide and silicone. Such materials are simple to manufacture in various shapes and sizes and are easy to sterilize.

In accordance with one aspect of the present invention, the stylet includes an internal power supply, such as a battery. In certain embodiments, such as when the light source and/or camera can function using very low electrical current, standard disposable dry cell batteries may be used to power both camera and lights. Batteries may be contained within the structure of the stylet, or located externally and connected via standard electrical connections. Some embodiments include a battery that may be replaceable, rechargeable, or disposable. Other embodiments may use an external power supply.

In some embodiments in which the stylet contains an internal power supply, the power supply may be activated in response to certain stimuli. For example, an embodiment may contain a data cable that can be extended and coupled to a display device, such as a video cable capable of delivering a signal viewable on a television screen. In such an embodiment, removal of the video cable may activate the power supply. Likewise, another embodiment may contain a port to which a separate video cable may be coupled. In that embodiment, coupling of the video cable to the port may activate the power supply.

In embodiments where power is supplied externally to the stylet, a standard electrical coupling may be used to transmit power from an external electrical source such as a battery or transformer. Visual signals are transmitted from the device's camera to a display screen, such as a liquid crystal display (LCD) or cathode ray tube (CRT), and such signals may be transmitted via a standard optical or electrical cables. Visual information may be stored in an analog or digital storage device for later retrieval.

The visualization stylet of the invention displays several advantageous characteristics including the fact that it is inexpensive to manufacture because it may be constructed from standard electrical components such as LEDs, CCD or CMOS cameras, and other standard electrical components. The cost of construction may be sufficiently small such that the device may effectively be disposable. If disposable, then the device requires no sterilization, reducing the cost of operation. The visualization stylet is also rugged and, because of its relative simplicity, is less prone to malfunction and damage than presently-used devices. Ease and effectiveness of use reduces the incidence of trauma to the patient and increases intubation speed, which may be life-saving. Additionally the stylet of the invention provides high quality optics and is easy to use without specialized training.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a schematic longitudinal cross-sectional representation of a general embodiment of the visualization stylet;

FIG. 2 is a schematic representation of the visualization stylet fitted within an endotracheal tube;

FIG. 3 is a schematic representation of a stereoscopic visualization stylet employing two separate cameras and two lenses;

FIG. 4 is a schematic representation of the visualization stylet of the invention fitted with an optional collimator;

FIGS. 5A and 5B are schematic representations of the visualization stylet of the invention fitted with an annular light source and a light transmissive element, respectively;

FIGS. 6A and 6B are, respectively, perspective and schematic representations of an embodiment of a visualization stylet employing an internal battery activated by attachment of a video cable;

FIGS. 7A and 7B are, respectively, perspective and schematic representations of an embodiment of a visualization stylet employing an internal battery activated by detachment of a video cable;

FIG. 8 is a schematic representation of the visualization stylet of the invention fitted with a wiper and sensors for detecting breathing;

FIGS. 9A and 9B are schematic and cross-sectional representations, respectively, of the visualization stylet of the invention fitted with manipulators in the stylet tube; and

FIGS. 10A and 10B are schematic and cross-sectional representations, respectively, of the visualization stylet of the invention incorporating electroactive polymer into the stylet tube.

### DETAILED DESCRIPTION OF THE INVENTION

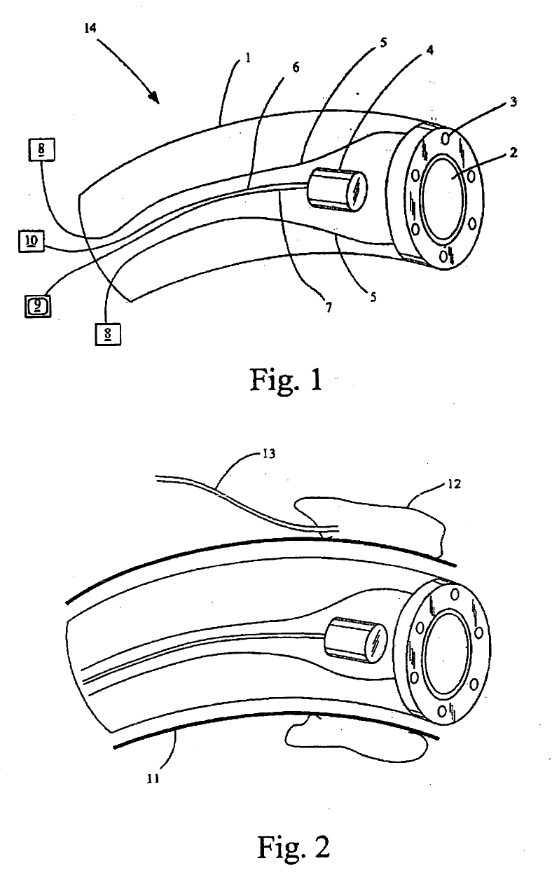

Referring to FIGS. 1 and 2, a schematic representation of visualization stylet 14 constructed in accordance with the principles of the present invention is described. All the elements in this particular embodiment of the stylet are contained within the lumen of stylet tube 1, although other embodiments may comprise additional features or elements in other locations. The stylet in this particular embodiment has a plurality of white LED lights 3 disposed in a circular pattern at the outside circumference of the distal tip of the stylet, surrounding central lens 2. The lens focuses light from an image onto CMOS camera 4. The LED lights receive power from one or more power conduits 5 that are electrically connected to power supply 8. The power supply comprise one or more dry cell batteries contained within the body of the stylet. The camera, which may be a CMOS or CCD camera, is centered within the axis of the lumen and slightly behind the distal tip of stylet tube 1, shielded from lights 3. The camera receives electrical power from power supply 10 via power supply conduit 6 and transmits visual information to video display 9 via data transmission conduit 7. The power supply to the camera and to the LED lights may be identical, depending on the voltage/power requirements of the camera and the LED lights.

The body of visualization stylet 14 preferably is formed from a hollow malleable tube. The stylet tube may be made of any suitable material that is plastic in nature, i.e., that maintains the shape into which it is bent. In a preferred embodiment the body is made out of a synthetic shape-retaining material. In general, aluminum, brass, plastic, or any other shape-retaining materials such as polyvinylchloride, polypropylene, polyethylene, polyester, polyamide, and silicone may be used.

The stylet may be straight or the distal portion of the stylet may curved. In a curved embodiment, the distal portion (approximately the distal 2 to 10 inches) may be evenly curved through an angle of between 2 degrees and 45 degrees, preferably between 5 degrees and 22 degrees, or between 7 degrees and 15 degrees. The portion of the stylet that is curved may be different for different anatomies, for example, for a baby, the stylet may be curved only at the terminal 1 to 3 inch portion. The maximum diameter of the stylet is appropriate so that it fits within the lumen of the endotracheal tube. For example, in an adult endotracheal tube having a diameter of 7.5 millimeters, a preferable diameter for the stylet of the present invention is approximately 6.5 millimeters. Likewise, stylets having smaller diameters are appropriate for pediatric endotracheal tubes.

Furthermore, the diameter may vary along the length of the stylet. In an embodiment with a non-uniform diameter, it is preferable to provide a stylet having a center section with a smaller diameter than the distal end in order to reduce the interaction between the exterior of the stylet and the interior of the lumen of the endotracheal tube or other device.

The distal tip of visualization stylet 14 includes one or more light sources 3. In a preferred embodiment, the light sources are disposed in a circular pattern at the outside circumference of the distal tip of the stylet. The light sources are preferably white LED lights, but may be incandescent or fluorescent lights or in another embodiment may be a non-coherent light source transmitted via a fiber-optic bundle. The light source may also comprise an annulus that either produces light, such as an LED, or transmits light from another light source. The one or more light sources project light forward from the tip of the stylet during intubation thereby illuminating the objects to be viewed. Incoming light rays reflected from the object to be viewed are focused through lens 2 onto camera 4.

In some embodiments, lens 2 or transparent facing 18 may be pretreated with a hydrophilic or hydrophobic substance to manage water, blood, or other substance that may be encountered during the intubation procedure and that may affect visibility. Other embodiments may manage these substances by use of lens 2 or transparent facing 18 formed from a hydrophilic or hydrophobic material. Some embodiments may employ mechanical devices to assist in maintaining visibility, such as a electroactive polymer (EAP) section that acts like a wiper blade on the surface of lens 2 or transparent facing 18. Although some embodiments position lens 2 directly in front of camera 4, other embodiments may utilize different configurations and may redirect light waves with mirrors or other known devices.

The camera is preferably a CMOS or CCD of a type commonly used in digital cameras. The camera receives power via power supply conduit 5 and transmits an electrical signal via data transmission conduit 7 to video display screen 9, such as an LCD or CRT screen. The operator views the screen to monitor the progress of the endotracheal tube through the vocal chords into the trachea.

The power and data-transmission conduits run within the lumen of the stylet and project out from the proximal end of the stylet, terminating in standard video output and power input couplings which are operatively attached to the video screen and the power source, respectively. If the device contains an internal battery, then only a video output need project from the proximal end of the device, as discussed in further detail below. Depending on the voltage requirements of the camera and LEDs and any other components, a single power supply (either internal or external battery) may be used to power the camera, LEDs, and other components. In a lesser-preferred embodiment where light is transmitted via fiber optic cables, a non-coherent fiber-optic bundle runs through the stylet tube from the light source to the distal tip of the stylet.

Internal battery 20 may be disposable, such as for a single-use application, or alternately may be rechargeable or replaceable, and therefore more appropriate for repeat stylet usage.

Still referring to FIG. 2, visualization stylet 14 is disposed within the lumen of endotracheal tube 11, with endotracheal cuff 12 deflated. The cuff is a flexible balloon toroidally attached about the outer surface of the distal end of endotracheal tube 11 and is in air/fluid communication with inflation tube 13. In use, endotracheal cuff 12 of endotracheal tube 11 is inflated by providing a positive pressure via inflation tube 13; endotracheal cuff 12 serves both to hold endotracheal tube 11 in place and to prevent passage of stomach or oropharyngeal contents into the lungs. Visualization stylet 14 may be used to ensure the proper positioning of endotracheal tube 11 prior to the inflation of endotracheal cuff 12. Visualization stylet 14 may then be removed from secured endotracheal tube 11.

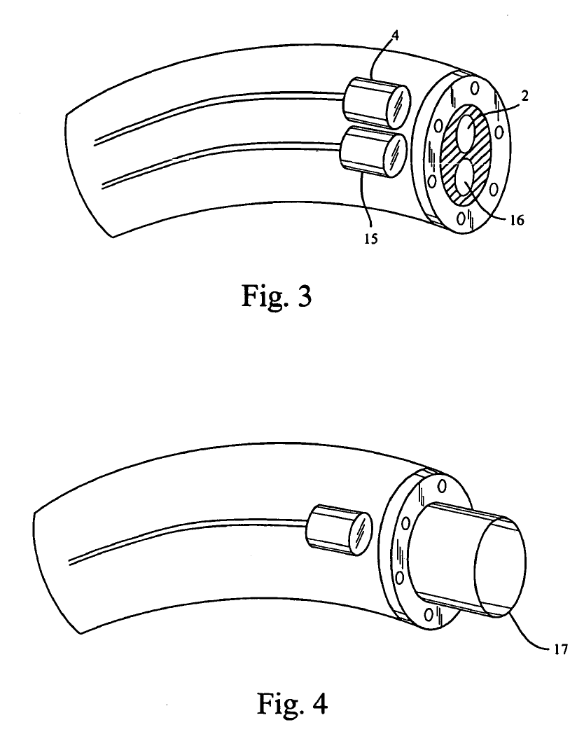

Referring now to FIG. 3, an alternative embodiment is described in which two cameras are mounted side by side to provide a stereoscopic image. In certain stereoscopic embodiments, lenses may be used to focus the light from objects into the cameras. The number of lenses will generally equal the number of cameras. In the embodiment of FIG. 3, there are two cameras and two lenses. In some embodiments, transparent windows may be provided in addition to, or in lieu of, the lenses to help prevent fluid and other matter from fouling the camera or other underlying components. Such a window may be made of glass or any other biocompatible suitable transparent material. In yet other embodiments, no lens or window is provided. Images are transmitted via the cameras and may be displayed on a screen using differential color imaging. Images also may be viewed by the operator using 3-D goggles to give the effect of a three-dimensional image.

Alternatively, the separate images may be processed by a computer to produce a three dimensional image that may be displayed and perceived without the need for special 3-D glasses. In another embodiment, a stereoscopic image may be provided without the need for a second camera. This may be done by splitting the single image into two images using an optical path separator and conducting each image to a separate camera. Such an embodiment may employ, for example, a single glass or plastic optical rod element to capture the initial single image, a prismatic optical path separator mounted behind the rod lens, and dual CCD OR CMOS elements to capture stereoscopic images. Video images can be processed electronically to convey images to a head-mounted display. See, e.g., Eguchi et al. “Stereoscopic Ophthalmic Microendoscope System,” Arch. Ophthalmol. 115:1336-1338, 1997 and Neurosurgical Focus 6 (4): Article 12, 1999, which are hereby incorporated by reference.

With respect to FIG. 4, an alternative embodiment is described that employs a collimator to shield the camera from being directly illuminated by the light sources. The collimator in the figure is somewhat exaggerated and need only be of a size and shape sufficient to shield the camera. In the example shown, the collimator is a hollow tube that projects from the distal tip of the stylet. The light sources (LEDs) are mounted circumferentially about the collimator, while the camera is positioned slightly back from the tip of the stylet and within the central lumen of the stylet tube. In other embodiments, separate collimators may be positioned over and around the individual light sources, forming a tube around the light that restricts the peripheral dispersion of the light so that only the desired target is illuminated.

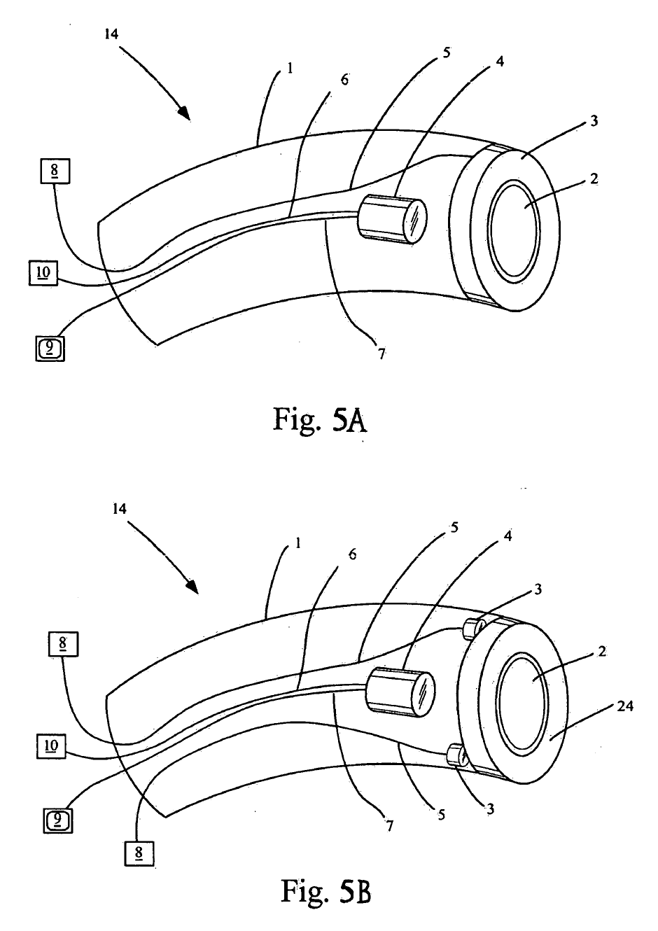

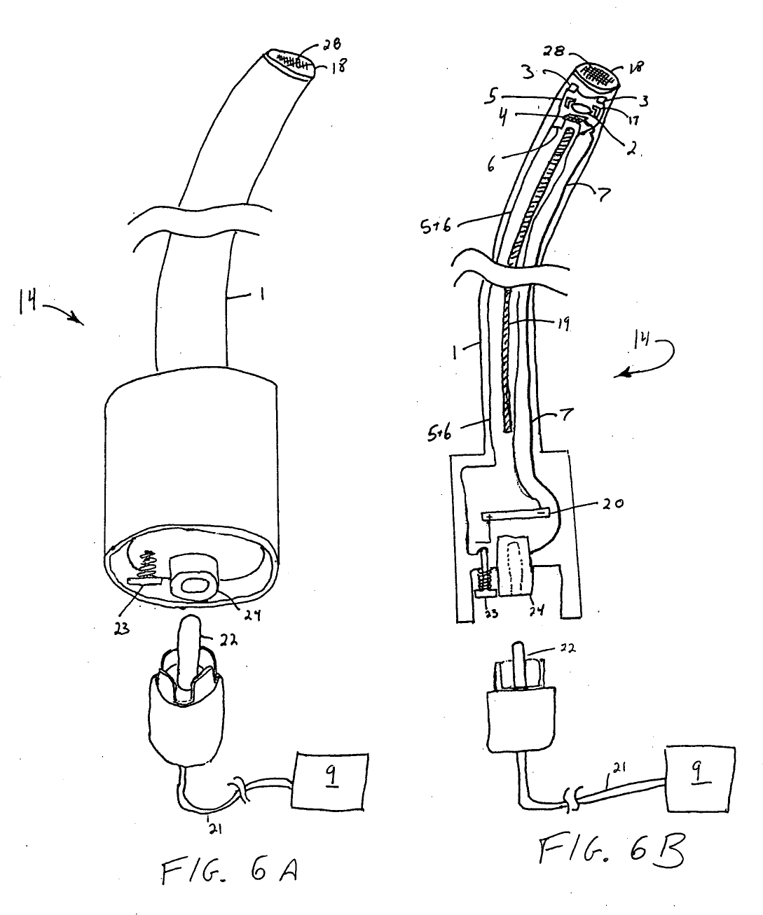

Referring now to FIG. 5, alternative configurations for one or more light sources are described for the stylet in accordance with the present invention. In FIG. 5A, light source 3 comprises an annulus or hoop-like structure disposed at the distal end of visualization stylet 14. Light source may comprise LEDs or other light emitting elements known in the art. Lens 2 is disposed within the interior portion of light source's 3 annulus. In this embodiment, lens 2 is formed from a hydrophilic or hydrophobic material to help manage water, blood, or other substances that may be encountered during the intubation procedure and that may affect visibility.

Light source 3 receives power from power supply for light 8, which is transmitted through power supply conduit for light source 5. Upon activation, annular light source 3 illuminates, thereby providing evenly-distributed light rays that may be reflected from the surrounding environment before entering lens 2 disposed within the central portion of light source 3. Advantageously, use of an annular light source 3 may allow for a reduction in the diameter of stylet 14, as the stylet's 14 distal tip may require less material for housing light sources 3 and other components.

Referring now to FIG. 5B, an alternative configuration for one or more light sources is shown. Here, light source 3 preferably comprises LEDs, but may also comprise fiber optics, incandescent light, fluorescent light, or other light source. In the embodiment shown in FIG. 5B, two light sources 3 are shown, although other embodiments may have more or less light sources.

Light source 3 is mounted adjacent to a light-transmissive annulus 24, such that activation of light source 3 illuminates light-transmissive annulus 24. Light-transmissive annulus 24 may comprise small non-coherent fiber optic bundles arranged in a ring shape or other known light-transmissive structures similarly arranged. Upon activation, stylet 14 operates as described above, in that the light is distributed substantially evenly around lens 2, which can then direct the reflected light rays to camera 4.

In use, visualization stylet 14 of the above-described embodiments is inserted into standard endotracheal tube 11 such that the tip of the stylet is at or near the distal tip of the endotracheal tube. Power supply conduit 5 (if both the camera and LEDs are powered by the same supply, which is preferable) or conduits 6 (if the camera and LEDs require a separate supply) and data transmission conduit 7 project from the proximal end of the endotracheal tube. The power supply conduit or conduits are operatively attached to appropriate power supplies (either internal battery, or external) and the data transmission conduit is communicably attached to a screen (e.g., LCD or CRT), thus providing a view of the patient's pharynx, glottis, and other anatomical structures during intubation. Once intubation is accomplished, the visualization stylet is withdrawn from the endotracheal tube and either sterilized for re-use, or preferably discarded.

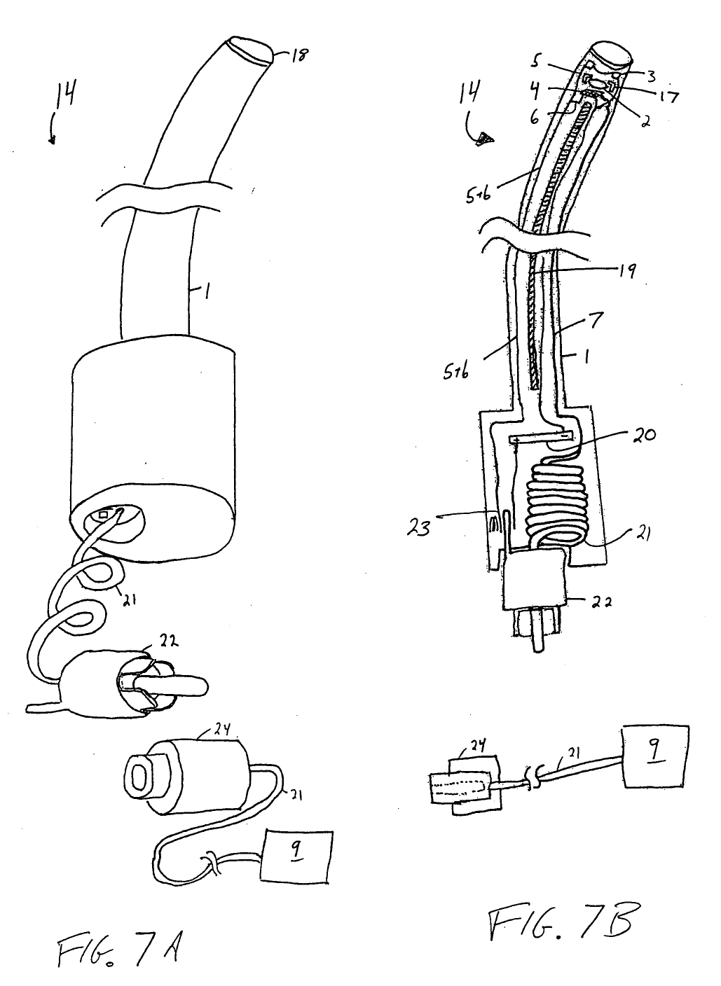

Referring now to FIG. 6, another embodiment of the present invention is described. Here, visualization stylet 14 is configured to activate an internal power source when attached to an external display source. In particular, visualization stylet 14 contains internal battery 20 that serves as power supply for light 8 and power supply for camera 10. Internal battery 20 is in communication with light source 3 and camera 4 via power supply conduits 5 and 6, which may coexist along at least a portion of their lengths, as shown in FIG. 6.

This embodiment further comprises stylet tube 1 that houses internal battery 20. In a stylet for use with adults, the overall length of stylet 14 is preferably about 40 cm, whereas pediatric stylets 14 are shorter. In this regard, when sized properly, stylet 14 should fit inside the lumen of an endotracheal tube (or other device), with a relatively small portion protruding from the endotracheal tube's (or other device's) proximal end.

In some embodiments, stylet tube 1, which preferably does not exceed a diameter of 6.5 mm, surrounds core 19, power supply conduits 5 and 6, and data transmission conduit 7. Core comprises a deformable structure, such as a thin metallic rod or similarly plastically deformable material, that may be manipulated into a variety of shapes by the user. Other embodiments may not have core 19. Camera 4, light source 3, lens 2, transparent facing 18, and collimator 17 are disposed near the distal end of stylet tube 1. In some embodiments, the exterior of transparent facing 18 or lens 2 may be covered with coating 28 to help manage water, blood, or other substances that may be encountered during the intubation procedure and that may affect visibility. Coating 28 may be a hydrophilic or hydrophobic substance. Yet other embodiments comprise transparent facing 18 or lens 2 formed of a hydrophilic or hydrophobic material.

Switch 23 is located along the communication path between internal battery 20 and powered components, here light source 3 and camera 4. In a preferred embodiment, switch 23 is biased in an open position until a user interacts with the device. In the embodiment depicted in FIG. 6, switch 23 comprises an element that moves when video cable male connector 22 is coupled with video cable female connector 24. Movement of switch 23 completes the electrical connection and allows light source 3 and camera 4 to receive electrical power. Other embodiments may comprise different known switching mechanisms. These mechanisms may be switchable between on and off positions, or may be one-way toggle switches that prevent unintentional deactivation by maintaining an “on” position after activation.

In the embodiment of FIG. 6, video cable 21 is attached to visualization stylet 14 using male connector 22 located at the distal end and female connector 24 located at the proximal end. The proximal end of video cable 21 is used as a source feed for video display 9. Preferably, male connector 22 and female connector 24 are commonly available connectors, such as RCA plugs and RCA jacks. The data output from stylet 14 preferably is in a format that may be directly delivered to video display 9, such as NTSC, PAL, or SECAM analog video signals.

The embodiment of FIG. 6 advantageously permits the physician to activate the device simply by connecting visualization stylet 14 to video cable 21 using the connectors. Preferably, the device is equipped with a 3 V or 5 V battery, such as a lithium coin battery, and provides approximately ten minutes of operating time prior to losing effectiveness. Similar embodiments also may employ various configurations of switches 23. For example, switch 23 may toggle the electrical connection by use of mechanical movement, magnetism, or other methods.

Referring now to FIG. 7, another alternative embodiment of the present invention is described. Here, the device is similar to the embodiment described above and in FIG. 6, but is shown in a configuration adapted to activate an internal power source when integrated video cable 21 is at least partially removed from visualization stylet 14. Likewise, whereas the embodiment described above and in FIG. 6 has coating 28 on the exterior of transparent facing 18, this embodiment comprises transparent facing 18 formed from a hydrophilic or hydrophobic material. Visualization stylet 14 contains internal battery 20 which serves as power supply for light 8 and power supply for camera 10. As discussed above, internal battery 20 may be disposable, replaceable, or rechargeable. Internal battery 20 is in communication with light source 3 and camera 4 via conduits 5 and 6, which may coexist along at least a portion of their lengths.

Switch 23 is located along the communication path between internal battery 20 and powered components, here light source 3 and camera 4. In a preferred embodiment, switch 23 is stable in a closed position, but is held in the open position until the user interacts with the device. As depicted in FIG. 7, switch 23 comprises an element that moves when male connector 22 is detached from stylet 14. Movement of switch 23 completes the electrical connection and allows light source 3 and camera 4 to receive electrical power. Switch 23 may be switchable between on and off positions, or may be a one-way toggle switch that prevents unintentional deactivation by maintaining an “on” position after activation.

In FIG. 7, video cable 21 is integrated into visualization stylet 14 and is configured to attach directly to a separate device having female connector 24. Preferably, male connector 22 and female connector 24 are commonly available connectors, such as RCA plugs and RCA jacks. The data output from stylet 14 preferably is in a format that may be directly delivered to video display 9, such as NTSC, PAL, or SECAM analog video signals.

This embodiment advantageously permits the physician to activate the device simply by pulling out male connector 24 and attaching visualization stylet 14 directly to a separate receiver with video display 9. Preferably, visualization stylet 14 contains approximately one meter of video cable 21 that may be extended from a compartment within the stylet. Preferably, the device is equipped with a 3V or 5V battery, such as a lithium coin battery, and provides approximately ten minutes of operating time prior to losing effectiveness. Similar embodiments also may employ various configurations of switches 23. For example, switch 23 may toggle the electrical connection by use of mechanical movement, magnetism, or other methods.

A preferred method of using visualization stylet 14 as depicted in FIG. 6 or 7 is now described. First, visualization stylet 14 is activated by either attaching video cable connector 22, such as for the embodiment of FIG. 6, or by removing video cable connector 22, such as for the embodiment shown in FIG. 7. Once visualization stylet 14 is activated, it may be inserted into standard endotracheal tube 11 such that the distal tip of the stylet is at or near the distal tip of the endotracheal tube. The power supply conduit or conduits are operatively attached to internal battery 20 and the data transmission conduit is communicably attached to video display 9, thus providing a view of the patient's pharynx, glottis, and other anatomical structures during intubation. Once intubation is accomplished, the visualization stylet is withdrawn from the endotracheal tube and preferably discarded.

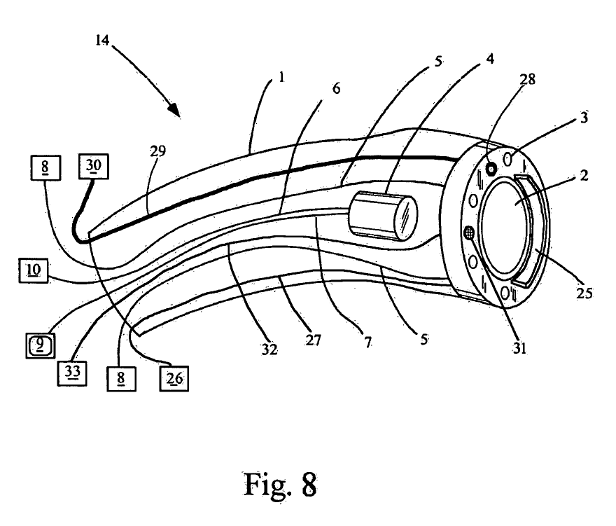

Referring to FIG. 8, a schematic representation of another embodiment of the visualization stylet of the present invention is described. In this embodiment, stylet tube 1 has a non-uniform diameter, a majority of which is smaller than the diameter at the distal end. Additionally, wiper 25 is provided to help reduce obstructions on lens 2. Here, conduit 6 connects camera 4 to power supply for camera 10, and output from the camera is communicated through data transmission conduit for camera 7 to video display 9. Light sources 3 receive their power from power supply for light 8, which is communicated through power supply conduits for light source 5.

Stylet tube 1 of FIG. 8 includes a non-uniform diameter along its length. In particular, the distal end of stylet tube 1 encircles lens 2, camera 4, and light sources 3. Proximal to this section, stylet tube 1 has a slightly narrower diameter than the distal end. Advantageously, this non-uniform design may reduce friction and the resistance encountered when moving visualization stylet 14 through a lumen of an endotracheal tube or similar device. Preferably, the reduced diameter portion is sufficiently rigid to allow visualization stylet 14 to be pushably advanced through a lumen without undesired kinking or flexing.

Another feature shown in the embodiment of FIG. 8 is wiper 25. Wiper 25 comprises a mechanical device to assist in maintaining visibility. For example, wiper 25 may comprise an electroactive polymer that moves across lens 2 or transparent facing 18 upon receipt of power sent from power supply for wiper 26 and communicated through conduit 27. Other wiper designs may include polymer blades or rotating surfaces.

The visualization stylet 14 also may comprise sensors that may be used to detect breathing. Two types of such sensors are illustratively shown in FIG. 8. First, carbon dioxide sensor 28 may be disposed at or near the distal end of stylet 14. Carbon dioxide sensor 28 is in communication with display 30 via CO2 sensor conduit 29. Display 30 may be integrated into stylet 14 or may be external. Alternatively, output from carbon dioxide sensor 28 may be delivered to video display 9. In this alternative embodiment, the output from carbon dioxide sensor 28 may be displayed in conjunction with the output from camera 4, or the video display may be switchable to selectively view the output from camera 4 or carbon dioxide sensor 28.

Alternatively, breathing may be detected using microphone 31 disposed at the distal end of stylet 14 and connected to speaker 33 via audio transmission conduit 32. Speaker 33 may be integrated as part of stylet 14 or may be external.

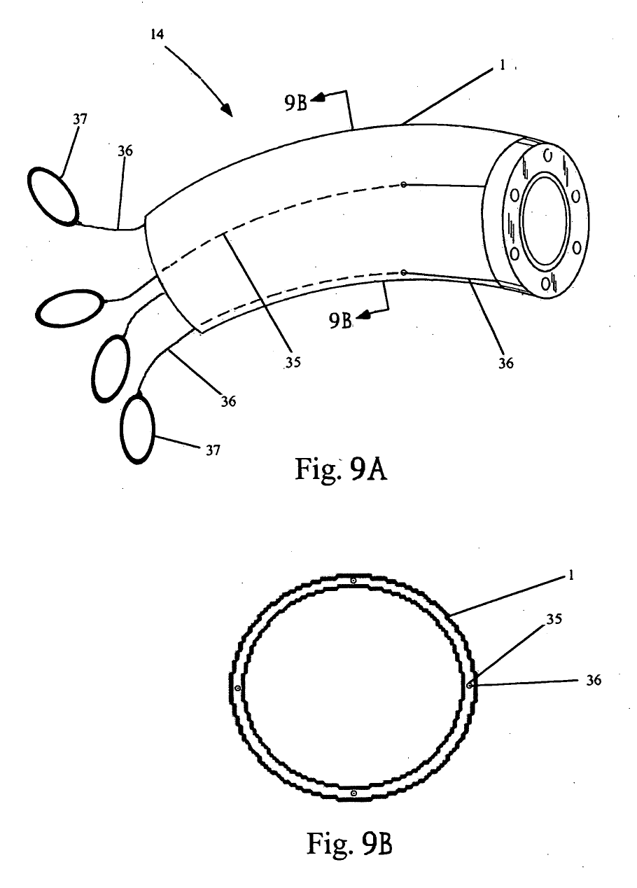

FIG. 9 depict an alternative embodiment of the stylet of the present invention. Here, stylet tube 1 comprises one or more lumens 34 through wall 35. Manipulator 36 passes through the lumen 34 and is affixed at the distal end. The application of force to the manipulator causes stylet tube 1 to deflect, thereby allowing steerage of the device without the need for removal and reinsertion.

Manipulator 36 preferably consists of wire, string, twine, or other flexible member capable of transmitting tensile force. Stylet tube 1 preferably comprises a polymer having sufficient flexibility such that tension applied to a manipulator causes deformation, yet sufficiently elastic such that stylet tube 1 may substantially return to an undeformed configuration in the absence of any outside forces.

Proximal end of manipulator 36 is configured to facilitate operation by the user, such as being attached to handle 37, lever, or other device. It should be appreciated that in some applications, such as an endotracheal tube, it is sufficient to allow manipulation in a single plane, such as simple up and down motion of the distal tip. Likewise, multidirectional motion may be desirable for other applications, in which stylet 14 may be moved through multiple planes.

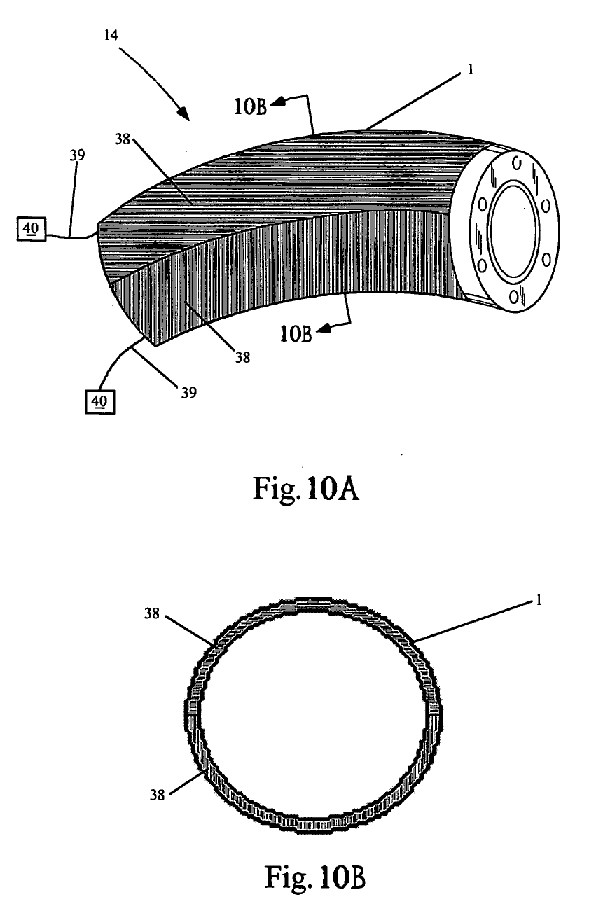

FIG. 10 depict an alternative embodiment of a stylet with a selectively deformable tube, in accordance with the principles of the present invention. In this embodiment, stylet tube 1 comprises electroactive polymer 38, activated by controller 40 through conduit 39. Accordingly, activation of electroactive polymer 38 by the user causes deformation of stylet tube 1, which may be used to steer the device without the need for removal and reinsertion.

In use, the embodiments shown in FIG. 9 or 10 are inserted into an endotracheal tube or other instrument and then the combination of devices is inserted into a patient. If the user determines that the feedback of the stylet 14 indicate that repositioning is desired, the user can then selectively deform stylet tube 1, such as by applying tension to a manipulator 36 or activating electroactive polymer 38. The user may then observe the output from stylet 14 to reevaluate the position if the devices, and continue to deform stylet tube 1 until the endotracheal tube or other surrounding instrument is in place.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. A stylet comprising:
an elongated body having proximal and distal ends and a lumen therebetween;
an image gathering device disposed within the lumen adjacent to distal end, the image gathering device electrically coupled to a receptacle adapted to communicate with an external unit;
a power supply disposed within the elongated body and electrically coupled to the image gathering device; and
a switch electrically connected to the power supply, the switch selectively permitting electrical connection between the power supply and at least one other component.

2. The apparatus of claim 1 further comprising a light source.

3. The apparatus of claim 2 wherein the light source comprises one or more light emitting diodes.

4. The apparatus of claim 3 wherein the one or more light emitting diodes comprises an annulus.

5. The apparatus of claim 2 further comprising light transmissive material at the distal end of the elongated body, wherein the light source illuminates the light transmissive material.

6. The apparatus of claim 2 wherein the power supply is a battery.

7. The apparatus of claim 6 wherein the receptacle is detachably mounted on the stylet.

8. The apparatus of claim 7 wherein the switch is activated when the receptacle is detached from the stylet.

9. The apparatus of claim 6 wherein the switch is activated when the external unit is attached to the receptacle.

10. The apparatus of claim 1 wherein the receptacle is an RCA plug or an RCA jack.

11. The apparatus of claim 1 wherein the switch comprises a spring.

12. The apparatus of claim 1 wherein the switch comprises a one-way toggle.

13. The apparatus of claim 1 wherein the body has a non-uniform diameter.

14. The apparatus of claim 13 wherein the diameter of the body is greater at the distal end than at a point between the distal end and proximal end.

15. The apparatus of claim 1 further comprising a lens disposed distal to the image gathering device.

16. The apparatus of claim 15 further comprising a coating disposed on the lens.

17. The apparatus of claim 15 in wherein the lens comprises a hydrophilic material or a hydrophobic material.

18. The apparatus of claim 15 further comprising a wiper disposed at the distal end of the body.

19. The apparatus of claim 18 wherein the wiper comprises electroactive polymer.

20. The apparatus of claim 2 further comprising a collimator optically disposed between the light source and the image gathering device.

21. The apparatus of claim 2 further comprising an audio receiver at the distal end of the body of the stylet.

22. The apparatus of claim 2 further comprising a carbon dioxide sensor at the distal end of the body of the stylet.

23. The apparatus of claim 22 wherein measurements from the carbon dioxide sensor are communicated to the external unit.

24. The apparatus of claim 2 wherein the elongated body is selectively deformable.

25. The apparatus of claim 24 wherein the elongated body further comprises one or more manipulators capable or transmitting tensile force, wherein the elongated body is deformed in response to tensile force applied to the one or more manipulators.

26. The apparatus of claim 24 wherein the elongated body further comprises electroactive polymer, wherein the elongated body is deformed in response to activation of the electroactive polymer.

27. A method of examining the interior of an opening comprising:
providing a stylet comprising a body having proximal and distal ends and a lumen therebetween, an image gathering device disposed adjacent to the distal end and adapted to communicate with an external unit, a receptacle detachably mounted to the stylet and coupled to the image gathering device, a power supply, and a switch configured to selectively couple the power supply to the image gathering device when the receptacle is detached from the stylet;
detaching the receptacle from the stylet;
inserting the stylet into an opening; and
receiving data from the image gathering device.

28. The method of claim 27 further comprising attaching the receptacle to an external unit.

29. The method of claim 28 wherein receiving data from the image gathering device comprises viewing images on the external unit.

30. A method of examining the interior of an opening comprising:
providing a stylet comprising a body having proximal and distal ends and a lumen therebetween, an image gathering device disposed adjacent to the distal end and adapted to communicate with an external unit, a receptacle electrically coupled to the image gathering device, a power supply, and a switch configured to selectively couple the power supply to the image gathering device when an external device is connected to the receptacle;
attaching the external device to the receptacle;
inserting the stylet into an opening; and
receiving data from the image gathering device.

31. The method of claim 30 wherein attaching the external device to the receptacle comprises providing communication between the external unit and the imaging device.

32. The method of claim 31 wherein receiving data from the image gathering device comprises viewing images on the external unit.
